(19) United States
(12) Patent Application Publication
(10) Pub. No.: US 2010/0135850 A1
(43) Pub. Date: Jun. 3, 2010

(54) AIR DISINFECTION DEVICE

(75) Inventor: Juha HELENIUS, Pori (FI)

Correspondence Address: YOUNG & THOMPSON, 209 Madison Street, Suite 500, Alexandria, VA, 22314, US

(73) Assignee: OY DESINFINATOR LTD., TAMPERE (FI)

(21) Appl. No.: 12/063,109

(22) PCT Filed: Aug. 4, 2006
(86) PCT No.: PCT/FI06/50351

§ 371 Date: Aug. 7, 2008

(30) Foreign Application Priority Data
Aug. 30, 2005 (FI) 20055458

(51) Int. Cl. A61L 9/00; H01J 27/02

(52) U.S. Cl. 422/4; 250/423 R

(57) ABSTRACT

A dry disinfection device comprising at least a process chamber (1), through which air is introduced into the environment. The process chamber (1) comprises both an ultraviolet radiation source (2) for producing ozone and an ionizer means (3) for ionizing air.

### FIELD OF THE INVENTION

The invention relates to a dry disinfection device according to the preamble of the appended claim 1 as well as a dry disinfecting method according to the preamble of the appended claim 7 and a dry disinfection unit according to the preamble of the appended claim 13.

### BACKGROUND OF THE INVENTION

A variety of methods can be used for the decontamination of air, including for example UV and filtering methods. Also ozone or negative ions can be used for the decontamination of air. Even though ozonization has been used for decades e.g. for the disinfection of tap water, it is rarely applied for the disinfection of air. Correspondingly, research references on the ionization of air are found beyond decades, but the application of the technique is still almost unknown. However, both of these methods are considerably more efficient than conventional UV and filtering methods.

In practice, ionization refers to the production of negative ions in the air. In nature, these ions are produced e.g. by cosmic radiation, radio-active radiation from the ground, UV light, charging caused by wind friction, electric discharges, combustion, and strong electric fields. In clean air, the lifetime of a negative ion is normally 100 to 1000 seconds. Negative ions are decomposed e.g. by such combustion processes in which particles are formed. For example, the smoking of one cigarette may reduce the ion concentration of a room to a level lower than one per mille of the starting level.

In the decontamination of air based on ionization, reactive oxygen species are supplied into the air to destroy various microorganisms and odorous organic compounds by oxidation. The ionization produces such reactive oxygen species which are not harmful to the human body. Consequently, ionization does not involve such concentration limits as ozonization. Another advantage of ionization is also the negative charging of particles in the air. Thus, the particles accumulate and adhere to surfaces, escaping from the air.

Ozone (O₃), in turn, is a triatomic form of oxygen with a strongly oxidizing property. in nature, ozone is formed e.g. by the effect of solar UV radiation in the upper atmosphere and, on the earth, for example in connection with lightning strokes. Ozone oxidizes several odorous compounds to an odourless form, and ozone is thus a good deodorizer. Furthermore, even low ozone contents have strong antiseptic properties. Even in small concentrations, ozone is very toxic to all viruses, anaerobic bacteria and fungi. Ozone may be used even against the MRSA (methicillin-resistant Staphylococcus Aureus) hospital bacterium which is fully sterilizable by using higher concentrations. It is easy to raise the ozone content temporarily to 1 to 3 ppm. For example, in the case of E. Coli, complete sterilization has been achieved at concentrations of 1 to 3 ppm in three hours. Resistant anthrax bacteria B. cereus and B. anthracis, in turn, have been completely sterilized by a 3 ppm ozone treatment for 48 hours. Even low concentrations of only 0.05 ppm may have antiseptic properties in long term if the ozone can be distributed evenly.

For people, long-term inhalation of large ozone contents causes damage e.g. in lung tissues, and therefore the ozone concentration must be limited. The allowed range for the ozone concentration varies generally from 0.05 ppm to 0.1 ppm. When high ozone concentrations (1 to 5 ppm) are used, one can stay in such a room only temporarily. However, ozone is a reactive compound that is degraded relatively fast, wherein the concentration of 1 ppm will drop to the allowed range in only a few hours, depending on the conditions. Therefore, efficient ozonization that is sufficient for sterilization can be performed, for example, after a working day, wherein the room is suitable for working on the next day. Another alternative is to decompose the ozone catalytically. By means of an atomizer, even an ozone concentration of 7 ppm will drop to the safe range in about 20 minutes. With appropriate equipment, such ozone decontamination can thus be performed in even urgent cases, or in places where e.g. operating theaters or other rooms with hygienic requirements are only vacant for short times.

Various devices have been developed for ozonization. Typically, the ozone is produced by UV light and distributed in the room by a fan. One such arrangement is disclosed in patent publication WO 2005/037409.

### SUMMARY OF THE INVENTION

The main purpose of the present invention is to provide a novel solution for dry disinfection, which enables the manufacture of a compact and effective air purifier to be used, for example, in hospitals and food industry.

To attain this purpose, the dry disinfection device according to the invention is primarily characterized in what will be presented in the characterizing part of the independent claim 1. The method according to the invention, in turn, is primarily characterized in what will be presented in the characterizing part of the independent claim 7. The dry disinfection unit according to the invention is primarily characterized in what will be presented in the characterizing part of the independent claim 13. The other, dependent claims will present some preferred embodiments of the invention.

The basic idea of the invention is to provide a dry disinfection device whose function is based on UV radiation, ozonization and ionization and which device is used to generate e.g. hydroxyl radicals (OH radicals) into the environment. The content of ozone released from the device into the environment can be advantageously kept low, wherein people can stay safely in the environment of the device.

In the solution according to the invention, a large quantity of negative ions is generated into the air. Upon an impact of oxygen atoms and the negative ions, superoxide radicals are formed. These superoxide radicals react with aqueous vapour in the air, forming perhydroxyl and hydroxyl radicals. Also according to the invention, ozone is generated into the same air. The production of hydroxyl radicals is accelerated further when the superoxide radicals react with ozone. The production of ozone and negative ions takes place closely in the same room. Thus, the different reactions of ozone and the negative ions take a time that is as long as possible. The device according to the invention releases hydroxyl radicals into its environment, as well as advantageously also negative ions and ozone. The radicals oxidize organic molecules strongly, thereby decontaminating the air. The negative ions and the ozone also decontaminate the air for their part. The forming hydroxyl radicals are among the most antiseptic compounds. For example in a deodorizing process, ozone and the radicals accumulate and decompose the organic compounds they detect, including e.g. odours. In the oxydizing reaction, the odorous substance turns to harmless carbon dioxide, aqueous vapour and oxygen.

The dry disinfection solution according to the invention makes very efficient decontamination of air possible even in rooms with people. Furthermore, the dry disinfection solution according to the invention provides many other advantages, including for example:
the decomposition and elimination of harmful particles and volatile organic compounds, and their conversion to a harmless form,
the elimination of odours,
the inactivation of microbes,
low energy costs,
minimized accumulation of particles onto surfaces,
no production of harmful reactants or side products.

Deodorization by UV radiation, negative ions and ozone is a very ecological way of keeping the air fresh. However, when ozone is used, one should bear in mind its toxicity in higher concentrations, wherein its content must be limited in continuous use. Nevertheless, with a solution based on ionization it may not necessarily be possible to sterilize a room completely, but the sterilization can be easily performed with high ozone contents. In one embodiment, it is thus possible to produce high ozone contents on a temporary basis. In one embodiment, excess ozone can be quickly decomposed, for example, by a catalytic atomizer after the sterilization.

By applying the solutions according to the invention, it is thus possible to implement various devices providing different features. These features include for example:
continuous ozonization of air and increasing it according to the need,
continuous production of negative ions,
option of quick decomposition of excess ozone,
efficient circulation so that both ozone and ions are distributed as evenly as possibly in the room.

This kind of a technique can be used not only in hospitals but also in households, in industry, service industries and in many other applications.

### DESCRIPTION OF THE DRAWINGS

In the following, the invention will be described in more detail with reference to the appended principle drawings, in which

FIG. 1 shows a dry disinfection device according to the invention in a cross-sectional view,

FIG. 2 shows a dry disinfection device according to the invention,

FIG. 3a shows a detail of the process chamber of FIG. 2,

FIG. 3b shows another embodiment,

FIG. 4 shows a dry disinfection arrangement installed in a ventilation duct.

For the sake of clarity, the figures only show the details necessary for understanding the invention. The structures and details that are not necessary for understanding the invention but are obvious for anyone skilled in the art have been omitted from the figures in order to emphasize the characteristics of the invention.

### DETAILED DESCRIPTION OF THE INVENTION

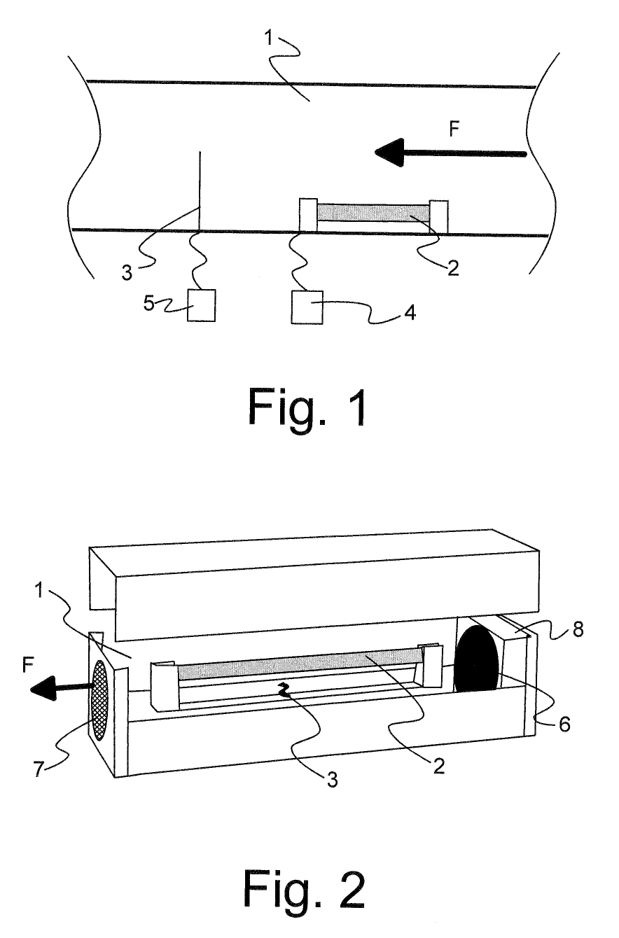

FIG. 1 shows a cross-section of a dry disinfection device according to the invention in principle. The cross-section is in the direction of the flow direction F of the air to be decontaminated, i.e. the device is shown from a direction perpendicular to the flow direction. The dry disinfection device comprises at least a process chamber 1 with an ozonizing means 2 and an ionizer means 3 which are controlled and input by suitable control and power units 4, 5. As the ozonizer means 2 used is advantageously an ultraviolet radiation source, the process chamber 1 is also called an ultraviolet chamber. One function of the process chamber 1 is to separate the ultraviolet radiation source 2 from the environment. Thus, the environment, including for example people, is not subjected to UV radiation. Furthermore, the process chamber 1 protects the ultraviolet radiation source 2 from external factors, such as e.g. dents. Advantageously, the process chamber 1 separates the environment from a direct contact with the ultraviolet radiation source 2, and suitable air inlet and outlet structures are provided to enable an air flow from the process chamber to the environment. The housing of the process chamber 1 can thus be implemented in a variety of ways while maintaining the basic idea of this invention.

The negative ions, OH radicals and ozone are produced inside the process chamber 1 to eliminate contaminants, moulds, viruses and bacteria effectively. For this reason, it is advantageous to use an ultraviolet radiation source 2 radiating in two wavelength ranges. The first wavelength is advantageously shorter than 200 nm, preferably 180 to 190 nm, and the second wavelength is longer than 200 nm, preferably 245 to 260 nm. In one application, the wavelength of 185 nm is used for ozone production and the wavelength of 253.7 nm e.g. for killing bacteria.

According to the invention, said ionizer means 3 for generating negative ions is also inside said process chamber 1. In FIG. 1, the ionizer means 3 is shown downstream of the ultraviolet radiation source 2 in the flow direction F of air. The ionizer means 3 may also be located upstream of, in parallel with or substantially in the same location as the ultraviolet radiation source 2. The ionizer means 3 and the ultraviolet radiation source 2 are, however, both in the same process chamber 1. Within the process chamber 1, air can flow advantageously freely between the ionizer means 3 and the ultraviolet radiation source 2; that is, there are no filters or fans between them. Advantageously, the ionizer means 3 and the ultraviolet radiation source 2 are located so that there are no obstacles between them. Thus, the different reactions of ozone and the negative ions, which will be described hereinbelow, take a time that is as long as possible.

The ionizer means 3 can be provided in several different ways. In one embodiment, a high-voltage discharge tip is used as the ionizer means 3. Typically, the voltage of the discharge tip 3 is 5 to 20 kV, and in one embodiment, the voltage is 10 kV. Advantageously, the discharge tip 3 produces almost continuously a large quantity of negative ions, wherein superoxide radicals are formed when some of the negative ions react with oxygen. The superoxide radicals react with possible aqueous vapour, forming perhydroxyl radicals and hydroxyl radicals which, in turn, may oxidize organic molecules. Furthermore, the super-oxide radicals also react with ozone, forming hydroxyl radicals and also hydroxyl anions.

The forming reactive oxygen species destroy various microorganisms and odorous organic compounds by oxidation. The above-mentioned radicals are also some of the most antiseptic compounds wherein, according to the invention, the decontaminating effect of the negative ions can be amplified significantly by the radicals at a very low ozone content that is safe for humans. According to the application, the device according to the invention can generate not only hydroxyl radicals but also negative ions and ozone to the environment. The negative ions and the ozone also decontaminate the air for their part.

FIG. 2 shows a device embodiment according to the invention with the process chamber 1 opened. In the exemplified device application, air is led into the process chamber 1 via an air inlet 6, and processed air is discharged via an outlet 7. To generate an air flow F, the device also comprises a fan 8 which is, in the example, on the side of the inlet 6. In the example, the ionizer means 3 is next to the ultraviolet radiation source 2.

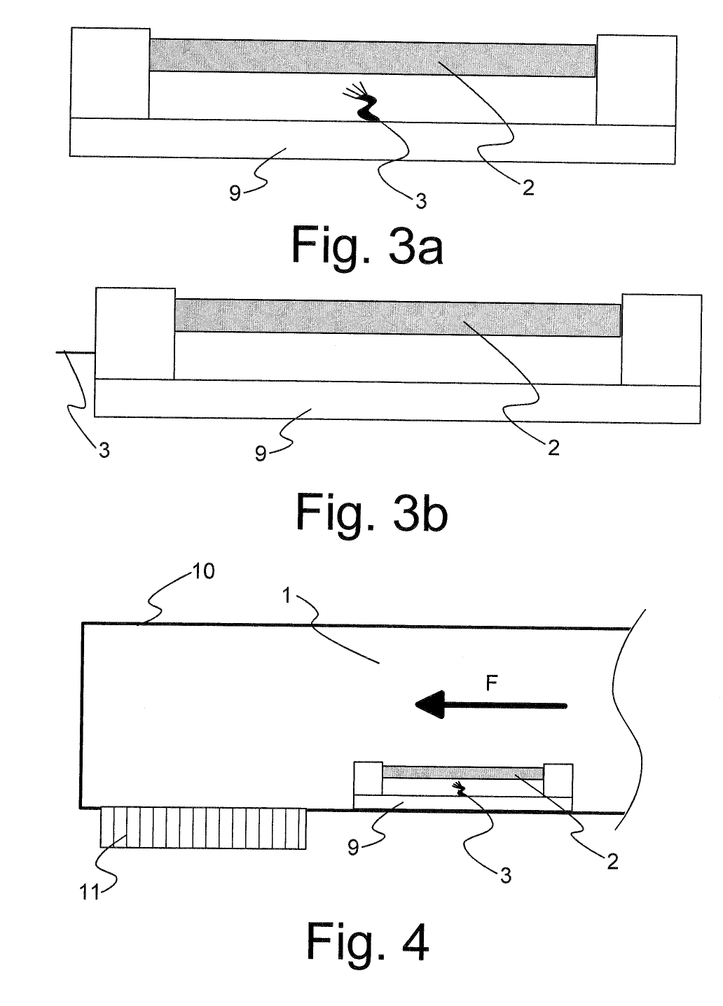

FIG. 3a shows a detail of the process chamber 1 of FIG. 2. FIG. 3 also shows the ultraviolet radiation source 2 and the ionizer means 3. In the example, the ultraviolet radiation source 2 is a UV lamp and the ionizer means 3 is a high-voltage discharge tip which, in the example, is a brush-like end of a flexible wire. Other suitable arrangements can also be used as the ionizer means 3. FIG. 3b shows an embodiment in which the ionizer means 3 is a discharge tip connected to a frame structure 9.

One embodiment of the dry disinfection device according to the invention can be made in such a small size that it can be carried by one person. A small device is also easy to position. Furthermore, the device according to the invention is easy to install, because the basic device only requires an electrical connection for its operation. Furthermore, structures requiring very little maintenance can be used for the ionization and ozonization. Typically, the lifetime of the ultraviolet radiation source 2 is about 10,000 hours, and the ultraviolet radiation source is, in practice, the only part of the device that wears in use.

In an advantageous embodiment, the control and power units 4, 5 shown in FIG. 1 are placed in the same frame structure in which the ultraviolet radiation source 2 and the ionizer means 3 are also arranged. The frame structure can be implemented in a variety of ways, and for example, it may resemble the frame structure 9 shown in FIGS. 3a and 3b. In one embodiment, the frame structure 9 comprises one connection point, through which electricity is supplied to both the ultraviolet radiation source 2 and the ionizer means 3. Such a structure makes a compact design possible and facilitates the assembly of various air decontamination systems.

FIG. 4 shows one application in which the dry disinfection device is arranged in connection with a ventilation duct 10. In the example, a part of the ventilation duct forms the process chamber 1 in which the ultraviolet radiation source 2 and the ionizer 3 are placed. In one embodiment, the installation of the ultraviolet radiation source 2 and the ionizer 3 is facilitated by the above-described integrated frame structure 9. Naturally, the ultraviolet radiation source 2 and the ionizer 3 may also be separate units. The air flow in the ventilation duct 10 is effected by a fan unit in the ventilation system. The ventilation duct 10 may comprise one or more outlets 11, depending on the application. The ventilation duct 10 may also supply air into two or more separate rooms, depending on the application.

In one embodiment of the invention, it is possible to produce high ozone contents. This makes a more efficient and/or faster sterilization of the rooms possible. The ozone content can be affected, for example, by controlling the ultraviolet radiation source 2 and/or by using several ultraviolet radiation sources that are turned on and off separately. In one embodiment, excess ozone formed by intensified ozone production is decomposed, for example, by a catalytic atomizer after the sterilization. The catalytic atomizer may be a part of the dry disinfection device or a separate unit.

In one embodiment of the invention, the dry disinfection device is supplemented with an air humidifier unit. Thus, the device also humidifies the air, and furthermore, the aqueous vapour, for its part, intensifies the formation of radicals.

By combining, in various ways, the modes and structures disclosed in connection with the different embodiments of the invention presented above, it is possible to produce various embodiments of the invention in accordance with the spirit of the invention. Therefore, the above-presented examples must not be interpreted as restrictive to the invention, but the embodiments of the invention may be freely varied within the scope of the inventive features presented in the claims hereinbelow.

1. A dry disinfection device comprising at least a process chamber, through which the air is introduced into the environment via an outlet, the process chamber comprising an ionizer means for generating ions and an ultraviolet radiation source connected to a frame structure for producing ozone, wherein the ionizer means, which is a high-voltage discharge tip, is also connected to the frame structure, and the ionizer means and the ultraviolet radiation source generate hydroxyl radicals to the environment via the outlet.

2. The dry disinfection device according to claim 1, wherein the device comprises a fan for producing an air flow into the process chamber, and that the process chamber consists of a housing comprising an air inlet and an air outlet.

3. The dry disinfection device according to claim 1, wherein the dry disinfection device is arranged to generate ozone, negative ions and hydroxyl radicals into the environment.

4. The dry disinfection device according to claim 1, wherein the discharge tip is supplied with a voltage of 5 to 20 kV.

5. The dry disinfection device according to claim 1, wherein the discharge tip is supplied with a voltage of 10 kV.

6. A dry disinfecting method in which air introduced into the environment is ionized in a process chamber by an ionizer means and subjected to ultraviolet radiation in the process chamber, to generate ozone, wherein the air is ionized by an ionizer means, which is a high-voltage discharge tip, connected to the same frame structure as an ultraviolet radiation source emitting ultraviolet radiation, and the ionizer means and the ultraviolet radiation source generate hydroxyl radicals (OH radicals), which are introduced into the environment.

7. The method according to claim 6, wherein at least hydroxyl radicals, ozone and negative ions are introduced into the environment from the process chamber.

8. The method according to claim 6, wherein the discharge tip is supplied with a voltage of 5 to 20 kV.

9. The method according to claim 6, wherein the discharge tip is supplied with a voltage of 10 kV.

10. A dry disinfection unit comprising at least a frame and an ultraviolet radiation source connected to the frame for the production of ozone, wherein the frame also comprises an ionizer means, which is a high-voltage discharge tip, for generating ions, and the ionizer means and the ultraviolet radiation source generate hydroxyl radicals to the environment.
